(19) United States
(12) Patent Application Publication
(10) Pub. No.: US 2005/0149097 A1
(43) Pub. Date: Jul. 7, 2005

(54) Transseptal needle

(76) Inventors: Sandra J. Regnell, San Jose, CA (US); Mark Forrest, Sunnyvale, CA (US); Raj Subramaniam, Fremont, CA (US)

Correspondence Address: HENRICKS SLAVIN AND HOLMES LLP;SUITE 200, 840 APOLLO STREET, EL SEGUNDO, CA, 90245, US

(21) Appl. No.: 10/749,773

(22) Filed: Dec. 30, 2003

(51) Int. Cl. A61M 29/00

(52) U.S. Cl. 606/191

(57) ABSTRACT

A transseptal needle and other structures and methods may include an outer conduit, for example one that may be subject to skiving, and a needle or other sharp structure within the outer conduit. A material no softer than the needle is positioned between the needle and the outer conduit so that the needle does not skive the inside surface of the outer conduit when the needle and outer conduit move relative to each other.

### BACKGROUND OF THE INVENTIONS

1. Field of Inventions

The present inventions are directed to methods and apparatus relating to the use of needles, including transseptal needles, and other structures and procedures for traversing tissue areas within areas of blood flow.

2. Related Art

Various procedures exist for accessing a blood flow area across a tissue wall for monitoring, diagnosis or treatment. In one example, though not the only situation where an instrument crosses a tissue wall into a blood flow area, a physician may want to access the left side of the heart from the right side by crossing a septum dividing the right atrium from the left atrium. The physician may be trying to access the tissue walls of the left atrium, heart valves on the left side of the heart, or other structures or regions on the left side of the heart. The physician may, for example, intend to form therapeutic lesions in the left atrium to treat cardiac conditions such as atrial fibrillation, atrial flutter and arrhythmia.

The heart chambers, as well as the vessels that carry blood to and from the heart, are shown in FIG. 1. The right atrium 12 pumps blood into the right ventricle 16, and the left atrium 14 pumps blood into the left ventricle 18. The atrial septum 20 divides the left and right atria 12 and 14, and the ventricular septum 21 separates the right and left ventricles 16 and 18. The tricuspid valve 22 allows blood flow from the right atrium 12 to the right ventricle 16 and prevents back flow, while the mitral valve 24 allows blood flow from the left atrium 14 to the left ventricle 18 and prevents back flow. The superior vena cava 26 and the inferior vena cava 28 open into the right atrium 12, and the pulmonary veins 30 open into the left atrium 14. The pulmonary artery 32 leads from the right ventricle 16 to the lungs and the aorta 34 leads from the left ventricle 18 to deliver oxygenated blood to the rest of the body.

Conventional methods and apparatus for delivering diagnostic or therapeutic elements to the heart by way of the femoral vein 40 are illustrated in FIGS. 2-8. Additional information concerning these methods and apparatus is provided in U.S. Pat. No. 5,575,810, which is hereby incorporated by reference. Referring first to FIGS. 2 and 3, an exemplary delivery system 44 includes an introducer 46 and an outer guide sheath 48. Both the introducer 46 and the guide sheath 48 are typically made from an inert plastic such as polyethylene. The distal end of the introducer 46 includes a skin-piercing cannula 50, which can be used to percutaneously access the femoral vein, while the proximal end includes a hemostatic valve 52 that blocks the outflow of blood and other fluids. The hemostatic valve 52 may be a conventional slotted membrane, a shutter valve arrangement, or any other structure that minimizes the outflow of fluids. The outer guide sheath 48 enters the introducer 46 through the hemostatic valve 52. The introducer 46 also preferably includes a flushing port 54 for introducing saline, anticoagulants or other fluids. Like the introducer 46, the guide sheath 48 is provided with a housing 58, which supports a hemostatic valve 62, and a flushing port 64. The distal portion 56 of the guide sheath 48 may have preformed curvature in some instances.

The delivery system 44 may also include a catheter 60 for directing the outer guide sheath 48 into the heart. The catheter 60 may be a steerable catheter, a catheter with a pre-curved distal tip, or any other catheter that can direct the guide sheath 48 to the desired region of the body. The exemplary catheter 60 illustrated in FIGS. 3-5 includes a catheter body 68 with a steerable distal tip 70 and a handle 72 with a steering mechanism 74. The exemplary steering mechanism 74 has a cam wheel 76, a knob 78 for rotating the cam wheel, and pair of steering wires 80 which are connected to the cam wheel and distal tip 70. The physician steers the distal tip 70 by manipulating the knob 78.

After the catheter 60 has been introduced into the guide sheath 48 by way of the hemostatic valve 62, the catheter body 68 and guide sheath may be advanced together through the femoral vein, while the housing 58 is kept near the catheter handle 72 to keep the catheter tip 70 beyond the distal end of the sheath. The physician will steer the catheter body 68 (and guide sheath 48) through the vasculature using the steering mechanism 74. Positioning of the catheter body 68 can be monitored with fluoroscopic or ultrasonic imaging or other conventional methods. When the catheter distal tip 70 reaches the right atrium 12, the guide sheath 58 will be advanced distally, from the location illustrated in FIG. 5, until the distal end of the guide sheath 48 is coextensive with the distal tip, as shown in FIG. 6. The guide catheter 60 may then be removed. Other delivery systems may also be used to place the guide sheath 48 in the right atrium.

As shown in FIGS. 7 and 8, the left atrium 14 can be accessed using a transseptal sheath assembly 82 that has been advanced through the delivery system 44 to the right atrium 12. The transseptal sheath assembly 82, which includes a transseptal dilator 82a and a transseptal needle 82b, may be positioned in the right atrium 12 against the atrial septum 20. The needle 82b is advanced relative to the dilator 82a to pass through the atrial septum 20 and into the left atrium 14. The dilator 82a is then advanced along the needle 82b into left atrium 14, thereby enlarging the opening formed in the atrial septum 20. The guide sheath 48 may then be advanced along the dilator 82a into the left atrium, thereby providing access to left side of the heart. The transseptal sheath assembly 82 may then be withdrawn from the guide sheath 48. It should be noted that the thickness of the atrial septum 20, which is a membrane sufficiently thin to allow relatively easy access from the right atrium to the left atrium, has been exaggerated in the drawings.

Advancement of the sharp needle tip through the dilator may cause particles to be skived from the interior surface of the transseptal dilator. Skiving can occur, for example, where the needle tip is metal and the interior surface of the dilator is plastic and where the dilator is curved. Any of these particles which may enter the bloodstream may pose a hazard as an embolus. Therefore, it is desirable to find ways to minimize the generation of particles by the needle tip.

### SUMMARY OF THE INVENTIONS

In accordance with one or more configurations of the needle combinations described herein, skiving or production of particles can be reduced, and methods can be used that reduce the production of particles in needle assemblies. For example, the potential for skiving can be reduced by incorporating needle assemblies that protect surfaces adjacent to the needle tip to minimize the production of particles through contact between the needle tip and adjacent surfaces. Such needle assemblies may be particularly useful when used with transseptal dilator tubes having a pre-formed curvature. Such assemblies can be used in other applications as well.

In one example of an assembly of a needle with another device, a dilator and a needle are combined such that the point of the needle is positioned within a distal portion of the dilator. The dilator may be a transseptal dilator and the needle may be a transseptal needle, for example. The needle is movable within the dilator and includes a first material configuration, for example hardness, surface smoothness, chemical composition and the like. A surface layer or surface segment is positioned between the dilator distal portion and the needle tip, and the surface layer is configured to have a second material configuration different than the first material configuration. The surface layer may be formed integral with the dilator or formed as a separate structure, for example a separate tube or lumen about the needle. The first and second material configurations are selected to minimize the possibility of skiving if the needle tip were to contact the dilator distal portion during normal operating procedures. For example, the material configuration may be hardness, and the surface layer hardness is greater than the needle hardness. The material configuration may be chemical composition, and the surface layer may be formed from stainless-steel and the needle tip formed from a reinforced plastic.

In another example of a needle assembly, a hollow element, for example a dilator, includes a sleeve movable within the hollow element. The sleeve is formed from a first material having a first hardness. A needle within the sleeve includes a tip formed from a material having a second hardness no greater than the first hardness. In one configuration, the sleeve protects the hollow element from the needle tip, thereby reducing possible production of particles that might occur if the needle tip were to contact an inside surface of the hollow element. In examples described herein, the needle may be a transseptal needle, such as one formed from hypotube. The needle may have a conventional pointed tip and hollow interior, for example for accepting a stylet and/or for measuring blood pressure. Alternatively, the needle may be closed ended, and may have side openings for passing fluid from the needle.

In an additional example of a needle assembly, the assembly includes a hollow element, for example a dilator, and a sleeve movable within the hollow element. A needle is positioned within the sleeve and is formed from a material no harder than the sleeve. The sleeve includes a distal end portion that has a converging surface portion, and may include a tapered distal end, a rounded end portion, or other similar distal end configuration. The sleeve may be segmented, for example with one segment harder than the other segment, with the harder segment being adjacent to the needle tip. The presence of the harder segment may reduce the likelihood of particles being created during movement of the needle.

In a further example of a needle assembly, the assembly includes a hollow element, for example a dilator, and a sleeve movable within the hollow element. A needle is positioned within a distal portion of the sleeve along with a positioning element at the distal portion of the sleeve for selectively positioning the needle. For example, the positioning element can be a spring or other bias for moving the needle forward to enter a tissue area, for example an atrial septum. The positioning element can also include a releasable latch, holding element or other structure for holding the needle in a disengaged or retracted position until such time as the needle is to enter the tissue area. A positioning element may allow the needle to be held stationary relative to the surrounding components while those components are maneuvered to the desired position, after which the needle can be moved into contact with the tissue area. This positioning element may also be located at the proximal end of the device, outside the body.

In another example of a needle assembly, the assembly includes a dilator having first and second segments and a needle within the dilator that is no harder than a hardness of one of the segments. In one configuration, the first segment is positioned, for example co-axially, between the needle and the second segment and the first segment is at least as hard as the needle. The first segment may be metal, a plastic, a powder injection molded portion and/or a reinforced portion, including one formed as a pultruded section. The first segment could be molded into the second segment or otherwise fixed to the second segment. In another configuration, the segments of the dilator may be positioned linearly, along the central axis of the dilator shaft. Here, the first segment is at a distal portion of the dilator and includes a curved portion along which the needle may move.

Needle assemblies can also be used in ways to reduce the possibility of generating particles. In one example of a needle and dilator combination, a transport tube having a portion formed from a first material may be introduced into the dilator, and a needle formed from a material no harder than the first material may be moved within the transport tube. Movement of the needle within the transport tube rather than along the surface of the dilator helps to reduce the possibility of skiving or otherwise generating particles. In one series of steps, the needle may be introduced into the transport tube prior to the transport tube being introduced into the dilator. Additionally, the needle tip may be kept within the transport tube until such time as the transport tube is positioned at the desired location relative to the dilator. In some configurations, all contact between the needle and the dilator may be precluded.

The above described and many other features and attendant advantages of the present examples will become apparent as the examples become better understood by reference to the following detailed description when considered in conjunction with the accompanying drawings.

### BRIEF DESCRIPTION OF THE DRAWINGS

Detailed description of preferred embodiments of the inventions will be made with reference to the accompanying drawings.

FIG. 1 is a simplified and diagrammatic longitudinal section of a human heart.

FIG. 2 is an exploded and partial cross-section view of a conventional catheter introduction system.

FIG. 3 is an exploded view of the catheter introduction system and partial cross-section view of the related anatomy illustrated in FIG. 2 with a conventional steerable catheter.

FIG. 4 is a side and partial cutaway view of a conventional catheter handle and steering mechanism.

FIG. 5 is a side and partial cutaway view of a catheter system and a venous pathway for gaining access to the right atrium of a heart.

FIG. 6 is another side and partial cutaway view of a catheter system and a venous pathway for gaining access to the right atrium of a heart.

FIG. 7 is a partial longitudinal section view of a human heart with a conventional transseptal needle in the right atrium.

FIG. 8 is a partial section, partial cutaway view of a dilator and transseptal needle assembly gaining access to the left atrium through an atrial septum.

FIG. 9 is a side view of a dilator and needle assembly in accordance with some of the present inventions.

FIG. 10 is a partial side-section view of the distal portion of the dilator and needle assembly illustrated in FIG. 9.

FIG. 11 is a side elevation view of a needle assembly in accordance with some of the present inventions.

FIG. 12 is an enlarged view of a distal portion of the needle assembly illustrated in FIG. 11.

FIG. 13 is a section view of a portion of the needle assembly illustrated in FIG. 11

FIG. 14 is a side elevation view of a shield that forms part of the needle assembly illustrated in FIG. 11.

FIG. 15 is a side elevation view of a needle forming part of the assembly illustrated in FIG. 11.

FIG. 16 is a partial section view of the distal portion of a dilator and needle assembly in accordance with some of the present inventions.

FIG. 17 is a side view of the distal portion of a needle assembly in accordance with some of the present inventions.

FIG. 18 is a section view of a dilator and shield in the form of an inner surface layer in accordance with some of the present inventions.

FIG. 19 is a section view taken along line 19-19 in FIG. 18.

FIG. 20 is an enlarged view of a distal portion of the dilator and inner surface layer illustrated in FIG. 18 in combination with a needle.

FIG. 21 is a section view of a dilator and shield in the form of an inner surface layer in accordance with some of the present inventions.

FIG. 22 is a section view of a portion of a dilator incorporating a needle in accordance with some of the present inventions.

FIG. 23 is a side, partial cutaway of a dilator incorporating a needle in accordance with some of the present inventions.

FIG. 24 is a side, partial cutaway view of the dilator illustrated in FIG. 23 with the needle extended.

FIG. 25 is a top plan view of an engagement mechanism for a needle combination.

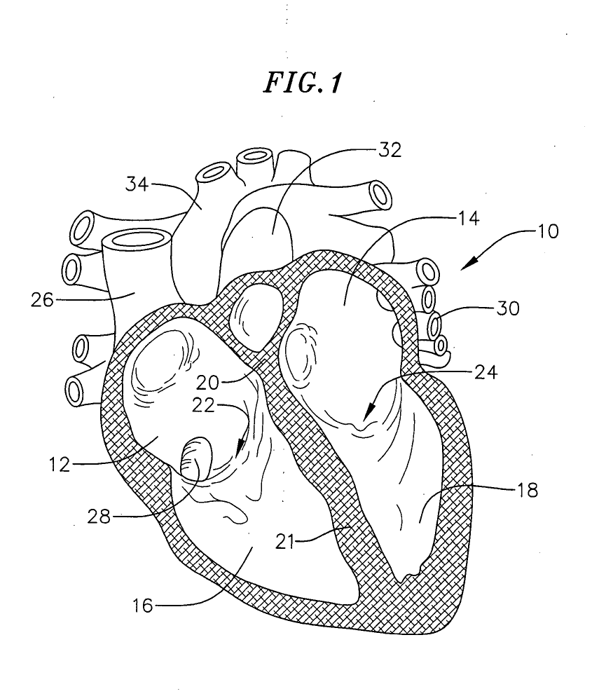

### DETAILED DESCRIPTION

The following is a detailed description of the best presently known modes of carrying out the inventions. This description is not to be taken in a limiting sense, but is made merely for the purpose of illustrating examples of apparatus and methods incorporating one or more aspects of the present inventions.

One or more aspects of the apparatus and methods described herein may be used within body lumens, chambers or cavities for diagnostic or therapeutic purposes such as, for example, in those instances where access to internal body regions is had through the vascular system, alimentary canal or other vessels without complex invasive surgical procedures. The apparatus and methods described herein may, for example, be used during the diagnosis or treatment of heart conditions. They may also have application in the diagnosis or treatment of conditions in other regions or organs of the body such as the prostate, liver, brain, gall bladder, uterus and other solid organs. The exemplary implementations described herein are presented as they may be used in conjunction with diagnosis or treatment of heart conditions, as they lend themselves to those applications.

Several examples of methods and apparatus that contribute to reducing occurrences of skiving when using a needle are described. In some examples, features or configurations of the apparatus or steps in a process are described together, for example because they are more efficient, simpler or otherwise more desirable when they are together. However, it should be understood that one or more benefits of the features or configurations may still apply when incorporated or used separately from the others. The described features, configurations or steps are presently-contemplated examples, but other examples with other features, configurations or steps or combinations thereof may also achieve one or more of the benefits of the present inventions.

Relatively sharp objects, for example needles, are used in many procedures in the human body. Needles are used in a wide variety of applications, and where the sharpened tip may come into contact with surfaces of other devices, for example the soft plastic of an adjacent lumen, the tip may skive particles from the plastic. Thereafter, the particles may enter the bloodstream and produce undesired consequences such as emboli which may obstruct blood flow anywhere in the body. Protection against contact between the sharp tip of the needle and adjacent objects helps to reduce the production of skived particles.

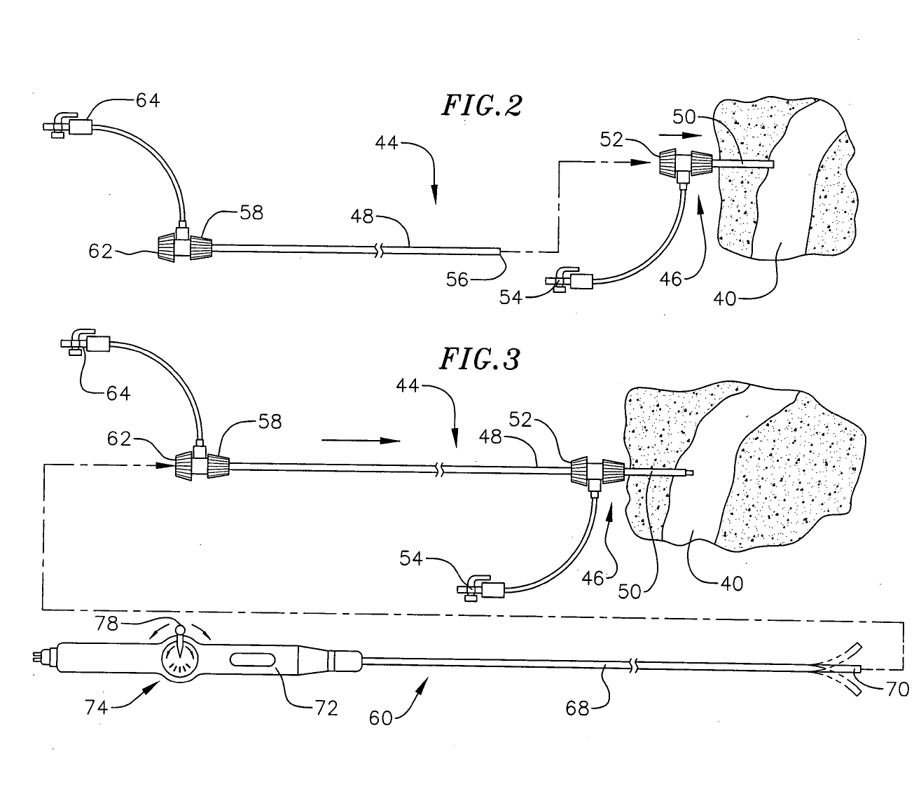

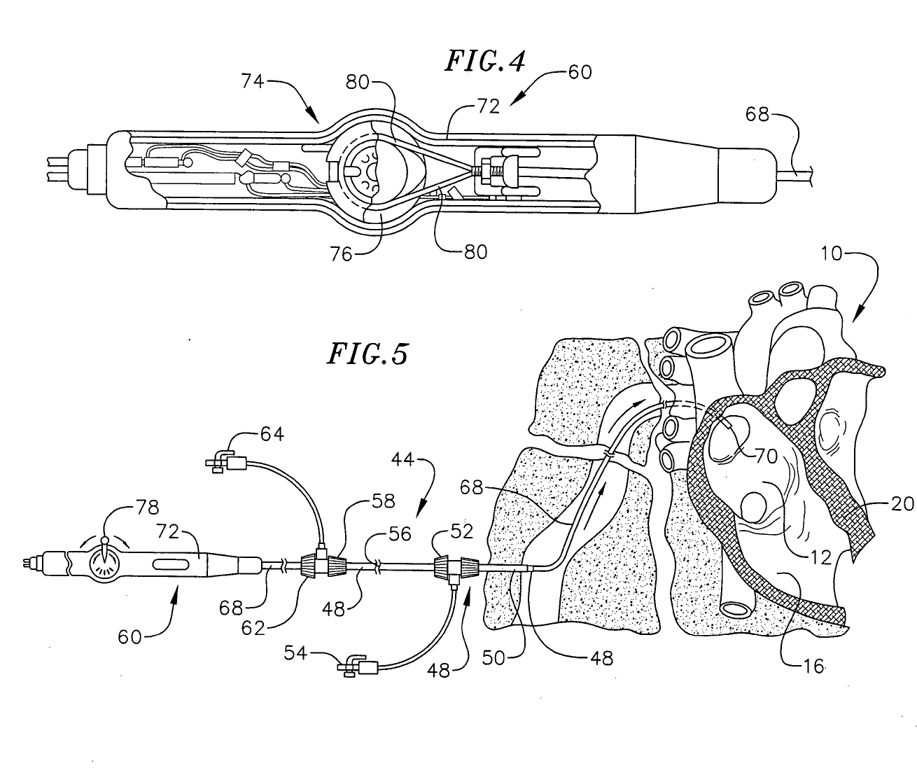

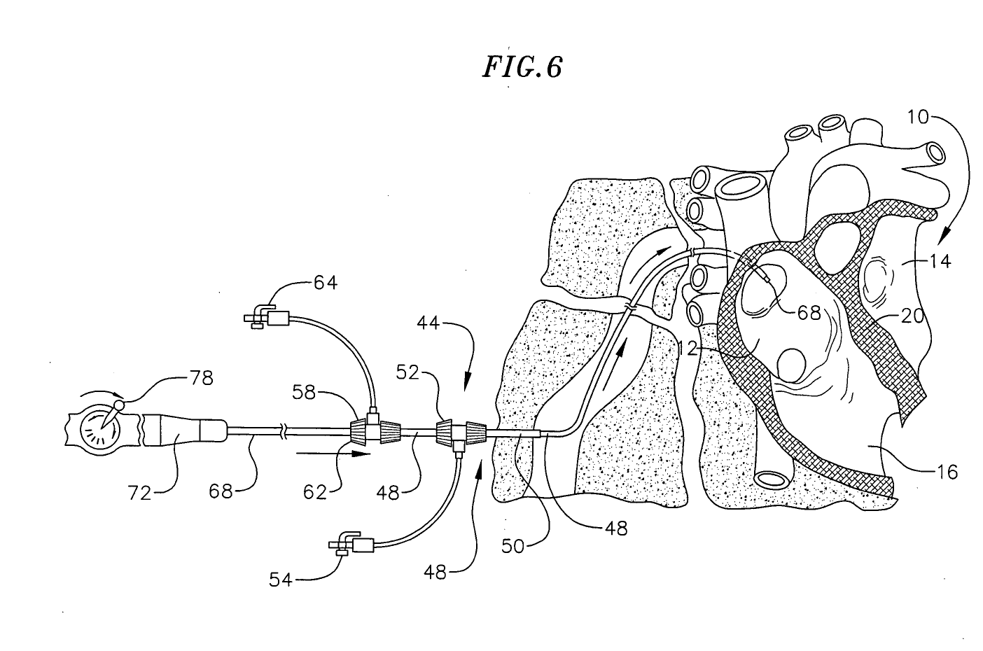

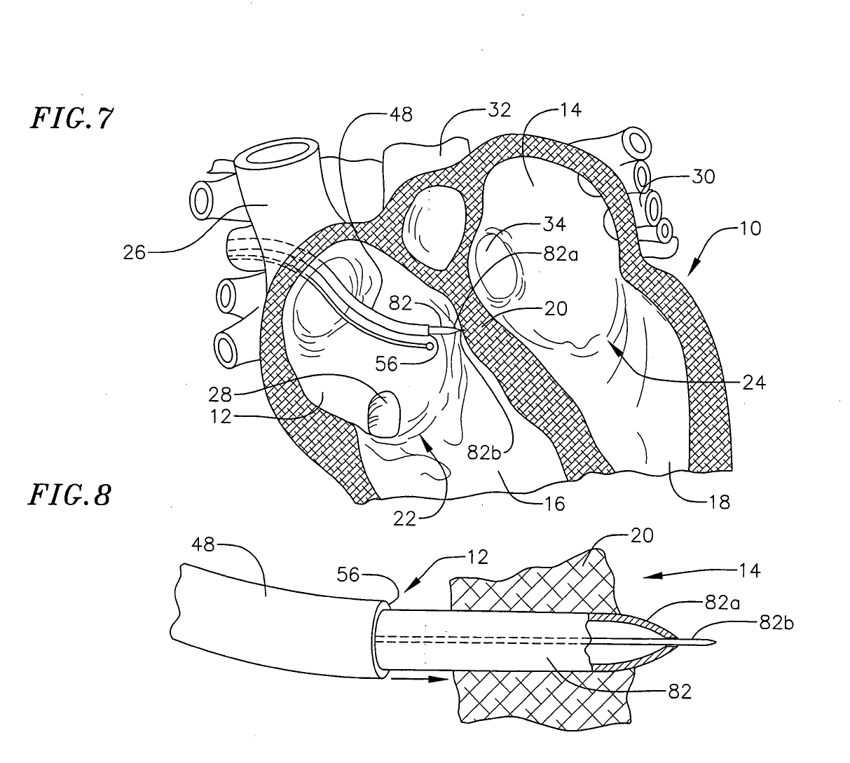

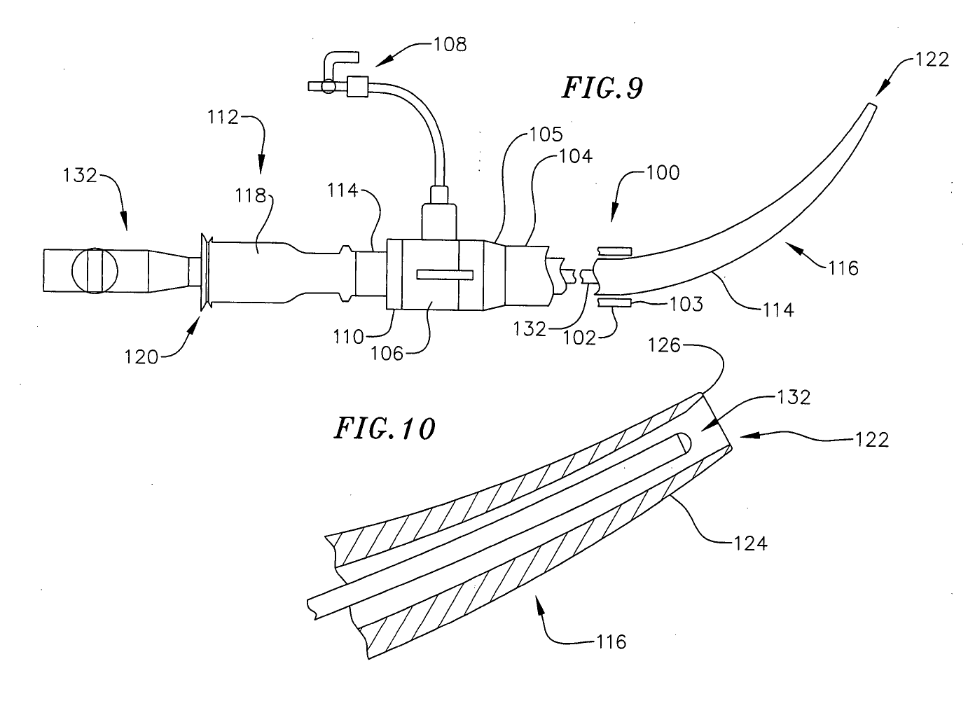

The present needle assemblies can be used with any suitable delivery system. Such delivery systems include conventional delivery systems that are sized and configured to be easily maneuvered and extended into the right atrium and thereafter into the left atrium. The delivery systems may also incorporate guidewires as well as steerable catheters. One example of such a delivery system is the delivery system 44 illustrated in FIGS. 2 and 3. Another exemplary delivery system, which is generally represented by reference numeral 100 in FIG. 9, has an elongate outer guide sheath 102 (with a structure and function similar to the aforementioned outer guide sheath 48) that may be introduced into the inferior vena cava by way of an introducer (such as introducer 46 shown in FIGS. 2 and 3). The exemplary sheath 102 has a curved distal portion 103 (the curvature is not shown in FIG. 9) that makes easier the positioning of the distal tip of the guide sheath and the distal end portion of the needle assembly adjacent to the atrial septum. The outer guide sheath 102 may be coupled at its proximal end 104 through a positive locking mechanism (such as a snap fit) 105 to a handle 106 used to manipulate and position the delivery system 100. The exemplary housing 106 is configured to accommodate a flushing port 108 and has a hemostatic valve 110 at its proximal end. The delivery system 100 will typically be formed from inert plastics or other materials that are suitable to medical applications.

As illustrated in FIGS. 9 and 10, an exemplary dilator 112, which may be advanced through the delivery system 100 and into the right atrium, includes an elongate dilator tube 114 with a distal portion 116 that has a pre-formed curvature to help in properly positioning the distal portion. The distal portion 116 also has an inner lumen or channel that is relatively constant in cross-sectional area over the length of the distal portion. The proximal end of the dilator tube 114 is fixed to a body 118 with a female luer hub 120. A guidewire (not shown) extending within the outer guide sheath 102 may be used to position the guide sheath distal portion 103 and dilator tube distal portion 116 within the right atrium. Once within the right atrium, the curved distal portions 103 and 116 may be manipulated into position adjacent to the atrial septum 20.

The curved distal portion 116 of the exemplary dilator 112 illustrated in FIGS. 9 and 10 terminates in a hollow distal tip 122 that may take a number of configurations. In the illustrated configuration, the distal tip 122 includes a gradually converging surface 124 that terminates in a transverse end surface 126, at which point the wall of the dilator tube 114 has a minimum thickness. The minimum thickness depends on the material from which the dilator tube is formed, and in one example, the minimum thickness of a polyethylene tube may range from approximately 0.003 to 0.005 inches, and a polyester elastomer such as Hytrel may have a minimum thickness of between 0.005 and 0.007 inches. However, with these materials and others, the minimum thickness can be similar or identical to the minimum thickness of conventional dilator tips. The dimensions, configurations and structure of the exemplary transseptal dilator tube 114 are otherwise the same or similar to conventional dilator tubes.

The exemplary dilator body 118 is provided with a suitable engagement surface, interlock or interference fit for securely receiving other components such as, for example, needle assemblies in the manner described below.

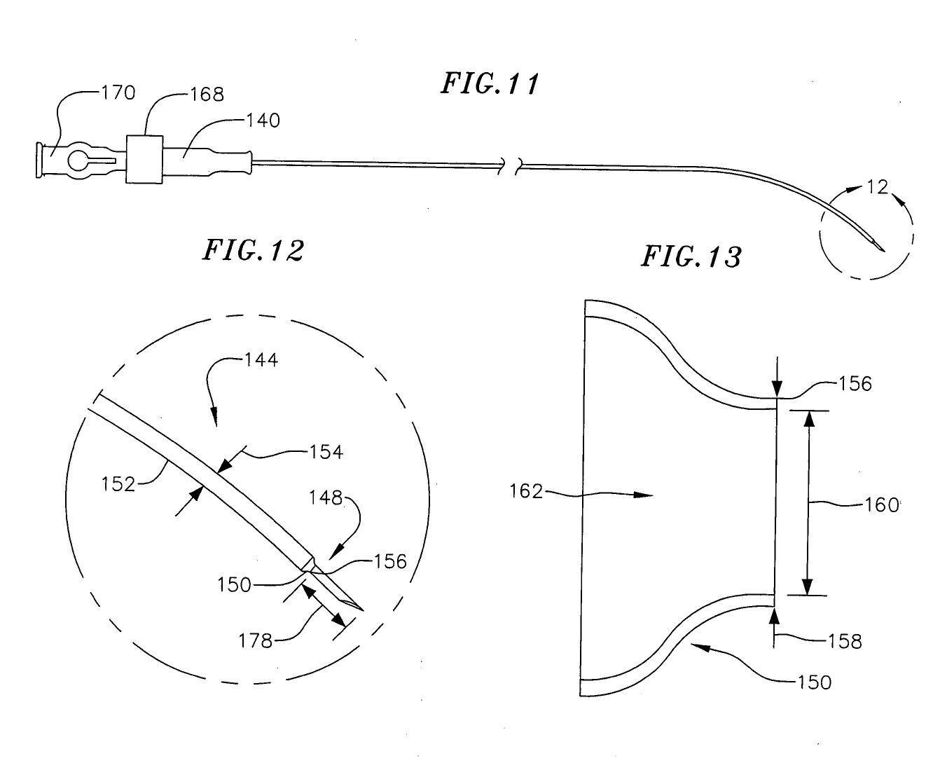

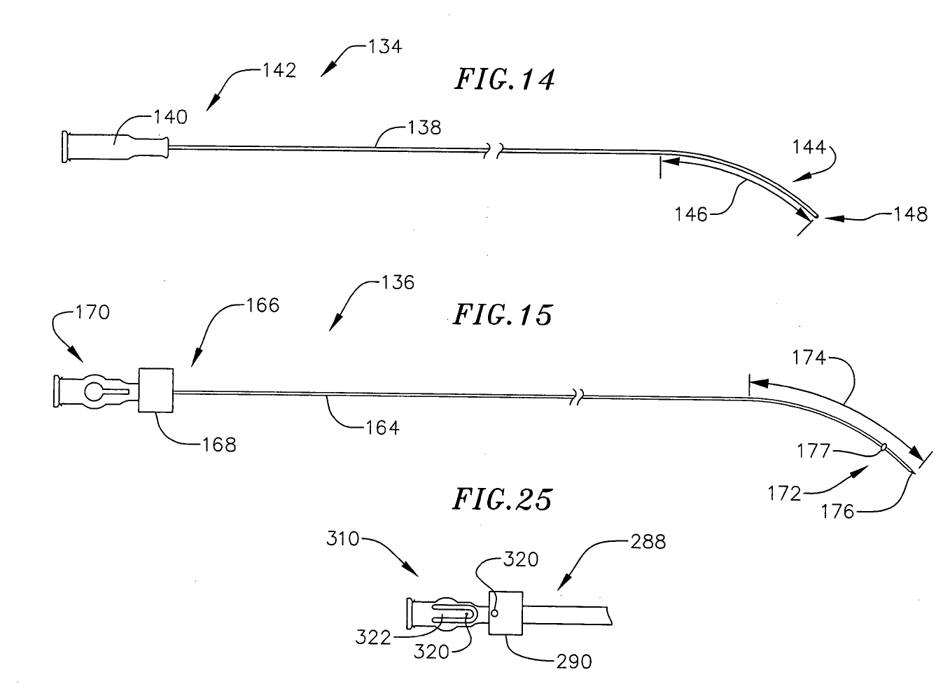

An exemplary needle assembly 132, which may be used with a dilator such as the dilator 112 to contact and operate on a tissue such as the atrial septum, is illustrated in FIGS. 9-15. While the needle assemblies described herein do not need to be used with a dilator, their application as transseptal needles typically include the use of a dilator, and the descriptions herein will be made in accordance with that particular use. The exemplary needle assembly 132 (FIG. 11) includes a shield in the form of an outside elongate and hollow shroud or sheath 134 (FIG. 14) and a separate elongate, and in this example hollow, needle element 136 (FIG. 15). The sheath 134 separates a sharp portion of the needle element from adjacent materials to reduce the possibility of skiving through contact between the sharp needle element and the adjacent material. Referring more specifically to FIG. 14, the exemplary sheath 134 includes an elongate hollow lumen or tube 138 coupled, attached or otherwise secured to a female luer hub 140 at a proximal end 142. The hollow tube 138 is accessible through the female luer 140 to, for example, receive the needle element 136 illustrated in FIG. 15. In the exemplary implementation, the hollow tube 138 has a distal portion 144 with a pre-formed curved segment 146 and terminates at a distal tip portion 148. The pre-formed curved segment 146 preferably conforms to the curvature of the curved distal portions 103 and 116 of the outer guide sheath 102 and dilator 112, respectively.

Turning to FIGS. 12 and 13, the distal tip portion 148 includes a convergent surface 150 extending from an outer surface 152 at an outside diameter 154 to a narrower rim surface 156 having an outside diameter 158 and an inside diameter 160. The inside diameter 160 is preferably sufficiently small to extend closely around the outside surface of the needle element 136 to minimize pinching of tissue between the two surfaces, while still permitting free movement of the needle through the opening 162 defined by the rim surface 156. The converging surface 150 may also be used to keep the tip of the needle element 136 separated from the surrounding material, for example the surrounding dilator surface, to reduce the possibility of skiving. The needle tip will never contact the inside surface of the dilator as long as the needle tip remains proximal of the rim surface 156.

The exemplary needle element 136 illustrated in FIG. 15 includes an elongate hollow needle shaft 164 attached, coupled or otherwise supported at a proximal end 166 to a male luer lock 168 and a stopcock 170 for controlling fluid flow within the needle shaft 164. The exemplary needle shaft 164 also includes a distal portion 172 that is preferably curved over region 174 to conform to the curvature of the associated dilator. The distal portion 172 preferably has a sharpened or pointed tip 176 to make easier entry into tissue or other material on which the needle is to be used. The distal portion also includes a shoulder or other enlargement 177 having an outside diameter larger than that of the adjacent distal portion of the needle. The outside diameter of the shoulder is also larger than the inside diameter of any distal opening through which the needle tip will extend to cross the atrial septum, for example the sheath tip 156. The shoulder provides an engagement surface that indicates that the needle should extend no further from the sheath, and preferably stops the needle from further distal motion within the surrounding sheath. The shoulder also preferably provides a close fit between the needle and the adjacent interior surface of the sheath when the needle is fully advanced within the sheath.

The shoulder is positioned a predetermined distance from the needle tip 176. The predetermined distance is chosen so that when the sheath is fully advanced within the dilator, and the needle is fully advanced within the sheath, the needle tip will traverse the atrial septum the desired distance, but not so far as to contact the opposite wall of the left atrium, or other adjacent tissue. The predetermined distance will typically be determined by a second distance. The second distance is from the mating internal wall of the sheath, with which the needle is used, to the tip of the dilator through which the needle tip 176 must extend. (The dilator is the dilator with which the needle and sheath assembly are used.) This second distance in turn is determined by the length of the converging portion 124 of the dilator and the length of the converging portion 150 of the sheath. Consequently, the size and location of the shoulder 177 are determined in conjunction with the configurations of the sheath and dilator with which the needle is to be used.

The needle element 136 (FIG. 15) extends within the sheath 134 (FIG. 14) to form the needle assembly 132. When the needle element and the sheath 134 are assembled, the needle tip 176 will preferably extend a distance 178 (FIG. 12) from the rim surface 156 a distance sufficient to extend about 0.5 to 0.75 cm from the distal tip 122 of the dilator.

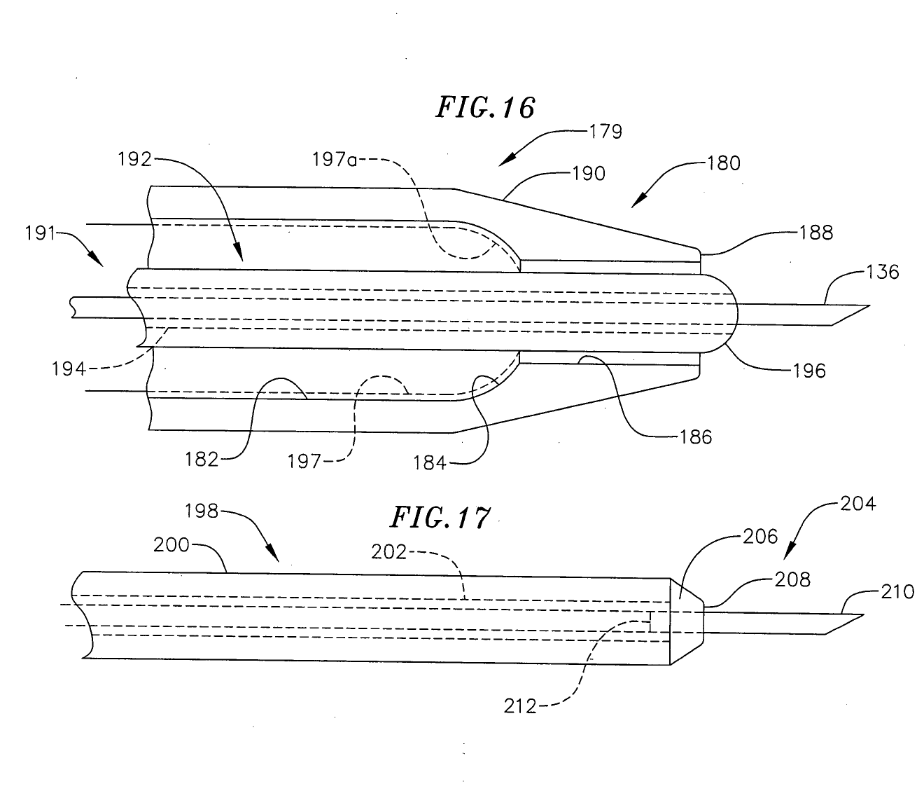

The shoulder 177 engaging the sheath is an indicator of the needle position relative to the sheath. Additionally, if the sheath is also fully within the dilator, the shoulder 177 engaging the sheath is an indicator of the needle position relative to the dilator distal tip 122. While it is not desirable to have unrestricted movement of the needle tip distally of the dilator tip, the shoulder 177 can be omitted if desired. Alternative to including a shoulder on the needle, other indicators of needle position and/or stop surfaces can be included on the proximal portions of the needle and/or the sheath. Other alternatives to indicating needle position can be used as well. Indicators can also be included on any of the other needles and/or outer sheaths described herein, including those in FIGS. 16 and 17.

In the exemplary implementation described with reference to FIGS. 9-15, the outer sheath or shroud 134 is preferably formed from tubing having a length equal to or longer than the associated dilator so that the outer sheath extends distally beyond the distal end of the dilator a distance sufficient to allow the needle element to function as desired. The tubing could have a wall thickness of between approximately 0.002 inch and approximately 0.015 inch. Preferably, the outside configuration of the outer sheath 134 matches very closely, at least at the distal end portion, the inside dimensions of the inside configuration of the dilator, so that the needle assembly moves easily within the dilator while still minimizing the possibility that the needle tip contacts the inside surface of the dilator. Where the outer sheath 134 is formed from thin walled tubing, the needle is preferably formed from the same material or a softer material, but sufficiently hard to minimize the possibility that the needle tip would become dull before it is used. By way of example, the tubing and the needle tip could be formed from hypotubing, thereby having the same hardness and material configuration. When formed from the same material, the adjacent surfaces of the needle and the sheath could easily have the same texture and finish, but could also have different surface characteristics. When formed from different materials, the surface finishes of adjacent surfaces could also be made the same, or they could be different.

As noted above, the exemplary needle assembly 132 may be used in conjunction with the dilator 112, which has a distal portion 116 with an inner lumen that is relatively constant in cross-sectional area. The needle assembly 132 may, alternatively, be used in combination with a dilator having a distal portion where the inner lumen dimensions are not constant. One example of such a dilator is generally represented by reference numeral 179 in FIG. 16. Here, an exemplary dilator distal portion 180 includes an internal bore defined by the internal wall surface 182 defining a first cross-sectional area, preferably circular in configuration, terminating in an internal converging wall or surface 184 having a gradually decreasing cross-sectional surface area. The converging surface 184 terminates in a preferably straight counter bore defined by a second internal wall surface 186. The second internal wall surface 186 preferably has a substantially circular cross-section that is constant from the converging surface 184 to a substantially flat rim surface 188 at the distal end of the dilator. The rim surface includes a fillet or radiused outer edge to provide a smooth transition to the converging surface 180.

When the needle assembly 132 is used in conjunction with the dilator 179, it is preferable that the converging surface 150 of the needle assembly distal tip portion 148 (FIG. 12) and the converging surface 184 of the dilator distal portion 180 (FIG. 16) be close fitting. In one embodiment, the dimensions are closely matched so that the spacing between the dilator first internal wall 182 and the outside diameter 154 of the sheath 144 is around approximately 0.001 to 0.005″ inch. For example, where the outside diameter 154 of the outer sheath is approximately 0.042 inch, the inside diameter of the dilator at the distal tip is approximately 0.047 at its maximum condition. That spacing can range from approximately 0.0005″ to approximately 0.0010″ inch if it is desired to have the surfaces substantially complementary to, for example, form a termination surface defining the distal-most position for the needle element. Additionally, the axial length of the surface 186 can range from approximately 0.25″ to approximately 1″, and will typically be about 0.50″. The outside configuration of the exemplary distal portion 180 illustrated in FIG. 16 includes a substantially straight converging surface 190. Alternatively, the converging surface may follow a more complex curvature. However, a straight converging surface or a more gradual converging surface, such as those associated with conventional dilators, is preferred. The dilator distal portion 180 may also include a pre-formed curvature or be substantially straight.

When the exemplary needle assembly 132 of FIGS. 11-15 is combined with dilator 179 of FIG. 16, the outer sheath 138 (FIG. 14) will not extend distally substantially beyond the converging surface 184. The close fit between the sheath converging surface 150 (FIG. 13) and dilator converging surface 184 stops the sheath at the converging surface. However, in other configurations of the dilator internal wall and the sheath outer dimensions, the wall terminating in the rim surface 156 (FIG. 13) can be extended to terminate within the counter bore defined by the surface 186, terminate flush with the flat rim surface 188, or terminate distally of the flat rim surface 188. In any case, it is still desirable for the needle to extend distally no further than about 0.5 to 0.7 cm beyond the distal-most structure adjacent the needle. As noted above, a number of structures and methods can be used to indicate when the needle tip has reached the preferred distal limit.

Another exemplary needle assembly, which is generally represented by reference numeral 191 in FIG. 16, includes the aforementioned needle element 136 and an outer sheath 192 that defines a lumen 194. The needle element 136 extends co-axially within the lumen 194. The outer sheath 192 has an outside diameter that is less than the inside diameter of the counter bore defined by the dilator wall 186. The outer sheath 192 terminates in an end surface. The end surface may be in the form of the converging surface 196 illustrated in FIG. 16, which has a radius. Other exemplary converging surface shapes include, but are not limited to, straight, elliptical, and parabolic. The proximal ends of the needle element 136 and outer sheath 192 are also preferably provided with the male and female luer locks described above with reference to FIGS. 11, 14 and 15. When the needle element 136 will have advanced to the fullest extent within the outer sheath 192, depending on the configuration, the distal end surface of the outer sheath 192 can extend distally beyond the flat rim surface 188 of the dilator, it can terminate within the counter bore defined by the surface 186, or it can terminate within the volume defined by the surface 184 or proximally thereof. In any case, the needle assembly is configured to permit the tip of the needle element 136 to extend beyond the surface 188 of the dilator. In the embodiments described herein, the distal tip of the needle element 136 preferably extends approximately 0.5 to 0.75 cm beyond the distal end of the distal-most one of the dilator and the outer sheath 134 or outer sheath 192.

In an alternative configuration of an outer sheath, intended for use with a dilator having a reduced surface dimension (for example, a reduced internal distal diameter), the outer sheath may include an enlarged body portion 197 (shown in phantom in FIG. 16). The body portion 197 includes a proximally located converging surface 197a for resting up against the correspondingly reduced diameter portion in the form of the converging surface 184 of the dilator. The needle assembly is advanced within the dilator until the sheath converging surface 197a contacts the dilator converging surface 184. The needle can then be deployed as desired.

The starting point on the outer sheath 192 of the transition to the proximal converging surface 197a can be selected as desired. In one configuration, the proximal converging surface 197a would simply be a continuation of the converging surface 196 at the end of the sheath. In this configuration, the outer sheath would remain substantially, if not entirely, within the dilator. In another configuration, proximal converging surface 197a would be spaced from the beginning of the converging surface 196, so that part of the converging surface 196 extends into the counterbore but still proximal of the rim 188. In another configuration, the proximal converging surface 197a is positioned so that the converging surface 196 is at the rim 188, and in another, the converging surface 196 is at least partly distal of the rim 188. It should also be noted that any of the needle assembly embodiments described herein may be used in conjunction with any of the dilator embodiments described herein. For example, the needle assembly 191 illustrated in FIG. 16 can be used in combination with the exemplary dilator 112 illustrated in FIG. 10.

The needle element 136 can take a number of configurations. For example, hollow, substantially cylindrical needle elements may be formed from hypotubes. Other configurations include solid access or puncture devices, and hollow tube elements having side access ports at the distal end portions which allow fluid to exit at least one side of the needle element. Hollow needle elements are preferred, however, because they permit the monitoring of blood pressure within the right and left atria. Blood pressure within the two atria are substantially different, and this fact can be used as evidence to confirm that a successful puncture of the atrial septum has been performed. In some preferred configurations, the needle element 136 is formed from a material that is no harder than the surrounding structure. With respect to the needle assemblies described above with respect to FIGS. 9-15, the needle element 136 is preferably formed from a material that is no harder than the material of the distal portion 144 of the outer sheath 134, and possibly no harder than the material of the dilator. In addition to hypotubes, needle elements may be formed from a reinforced plastic, a pultruded material, a glass or carbon reinforced plastic, reinforced ABS, or high impact ABS or any other plastic material capable of maintaining a sharp tip and transmitting the force required to puncture the septal wall.

Sheath 134 can be made of polypropylene, polystyrene, polyester or any other material suitable for producing a thin walled configuration with enough structural integrity to maintain a relatively rigid sheath while resisting scoring from the needle.

Another exemplary needle assembly is illustrated in FIG. 17. Needle assembly 198 includes an outer, elongate tube, shroud or sheath 200 (only the distal portion is visible) with a hollow substantially cylindrical internal wall 202 defining a lumen that receives a needle element 204. The internal wall 202 is preferably substantially circular in cross-section and extends the full-length of the sheath 200. The exemplary sheath 200 also includes a straight converging surface 206 with the external shape of a truncated cone, whose terminal edges are relatively smooth or rounded. The converging surface 206 terminates in a preferably flat, curved-edged end surface 208, which defines the opening through which the distal portion of the needle element 204 extends to contact tissue. The spacing between the needle element 204 and the sheath 200 around the opening is preferably sufficient to allow free movement of the needle element relative to the sheath. The sheath converging surface 206 will contact a similar converging surface on the inside of a dilator in which the needle assembly is placed. Alternatively, an additional protruding surface may be placed proximally for contacting a converging surface inside a dilator to limit the distal advance of the needle assembly. The needle element 204 will ultimately be advanced the desired distance to penetrate the atrial septum.

The exemplary needle element 204 illustrated in FIG. 17 is a two-part structure that includes a distal tip 210 and a proximal part 212. The distal tip 210 may be a cylindrical segment extending proximally a millimeter or more to the proximal part 212. The length of the distal tip 210 preferably ranges from approximately 0.5 to 1 cm, depending on where the sheath converging surface 206 seats relative to the end of the dilator. The transition area between the distal tip 210 and the proximal part 212 is preferably within the interior of the sheath and unexposed to tissue. The distal tip 210 is preferably formed from a material no harder than the dilator material, and can be formed from a number of materials, including metal, plastic, reinforced plastic, including metal, glass and carbon reinforced plastic, and other materials. The proximal portion 212 extends proximally from the distal tip 210 a distance sufficient to allow the needle element to be manipulated as desired. The proximal portion 212 can also be formed from a number of materials, including the materials from which the distal tip 210 may be formed. Additionally, the distal tip and the proximal portions of the needle element may be formed from the same material. The distal tip and the proximal portion are secured together using conventional techniques.

The parts (or segments) that form the exemplary needle element 204 are arranged longitudinally with respect to each other. In other exemplary embodiments, the needle parts can be arranged concentrically and/or circumferentially with respect to each other, or arranged partly longitudinally and partly circumferentially with respect to each other. The parts can have the same characteristics or characteristics that are slightly or significantly different from one another. Such differences may include, for example, different hardnesses, different thicknesses, different flexibility, and different surface characteristics (e.g. smoothness, texture, and slip characteristics). The parts may also have different dimensions and/or shapes. It should also be noted here that any of the needle elements described herein may be formed as a unitary (i.e. one part) structure, as a two-part structure, or as a structure with more than two parts.

Systems consisting of some or all of the aforementioned components may, for example, be used in the exemplary manner described below to obtain access to the left atrium. After the components are inspected and flushed, percutaneous access to the femoral vein is obtained by way of an introducer (such as the introducer 46 illustrated in FIGS. 2 and 3). While monitoring intra-chamber pressures and maintaining conventional fluoroscopy, a guide sheath 102 and transseptal dilator 112 are introduced over a guidewire into the right atrium, and the guidewire and dilator are removed. Other means may also be used to introduce the guide sheath. Outside the patient, a needle assembly 132 is inserted into the dilator 112, and the dilator irrigated. The needle and dilator are advanced with the needle tip unexposed into the right atrium and, when the dilator tip is near the distal end of the guide sheath 102, the combination is positioned near the atrial septum, for example by rotating the guide sheath, dilator and needle as a unit, clockwise until the conventional indicator on a proximal portion of the needle is in the 3-6 o'clock position. The guide sheath is withdrawn sufficiently to expose the dilator, and the dilator and needle are withdrawn sufficiently so that their distal tips are just below the lip of the atrial septum (fossa ovalis). When they jump posteriorly, the needle and dilator are advanced slightly until resisted. Once the proper positioning at the atrial septum is confirmed, the needle element 136, and the outside sheath 134 if desired, are advanced to penetrate the atrial septum. The dilator 112 is then advanced across the septum, over the outside sheath 134, after which the outside guide sheath 102 may be advanced across the septum the desired distance. The needle assembly 132 is then retracted into the dilator 112, and the dilator and needle assembly withdrawn together from the introducer. Other steps can be followed, and the sequence of the steps may be varied. For example, the outside sheath 134 can be retained interior to the distal end of the dilator 112. If desired, only the needle element 136, the dilator 112 and the outer sheath 102 will contact the atrial septum. The puncture can also be made during the process of inserting the needle combination into the dilator, followed by retraction of the assembly as the dilator is advanced through the puncture. This sequence of steps can be done without having the needle and outer sheath axially fixed to each other, such as through a retention mechanism.

Other methods can be used to introduce the dilator and needle to the right atrium. Methods used include advancing a guide catheter from the femoral vein through the inferior vena cava to the right atrium. The guide catheter can include the dilator and needle as it is being advanced into the right atrium, or the dilator and needle can be advanced into the right atrium once the guide catheter has entered the right atrium. Another method uses a steerable catheter to advance a guide catheter into the right atrium. The steerable catheter is removed, and the dilator and needle are advanced through the guide catheter and located adjacent the atrial septum. Approaches other than the femoral vein can also be used.

As the dilator and needle combination are advanced to the atrial septum, the needle tip is preferably kept inside the distal tip of the dilator. In that condition, the needle tip can be positioned at a number of places relative to the outer sheath, as described previously. Relative to the dilator distal tip, the needle tip is preferably within the dilator distal tip, but may be positioned between the dilator tip and the outer sheath tip, flush with the outer sheath tip or proximal of the outer sheath tip. When the needle tip is either flush with or proximal of the outer sheath distal end, the outer sheath still can be distal of the dilator while protecting the needle tip. Once the outer sheath and needle are positioned at the atrial septum as desired, the needle can be advanced to traverse the septum. If the needle is advanced from inside the dilator, the outer sheath 134 can be retained interior to the distal end of the dilator 112, or can be advanced outside the dilator with the needle element 136, as the needle element penetrates the atrial septum. The dilator 112 may then be advanced across the atrial septum, followed by the guide catheter.

Whether using the pre-formed curved dilator and needle assembly or the steerable catheter, use of a surface layer, segment or shield, for example in the form of a shroud or sheath, between the tip of the needle and the dilator helps to reduce skiving. Forming the needle tip from a material no harder than the material of the shield contributes to reducing skiving. For example, forming the shield and the needle tip from hypotube is convenient and forms a sufficient protection between the needle tip and the adjacent dilator surfaces.

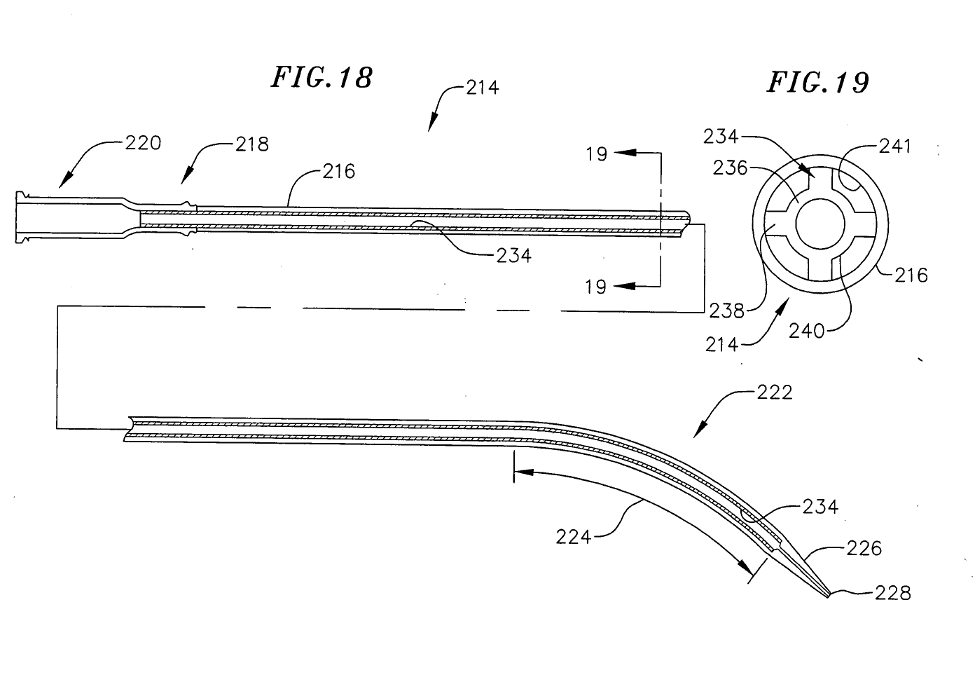

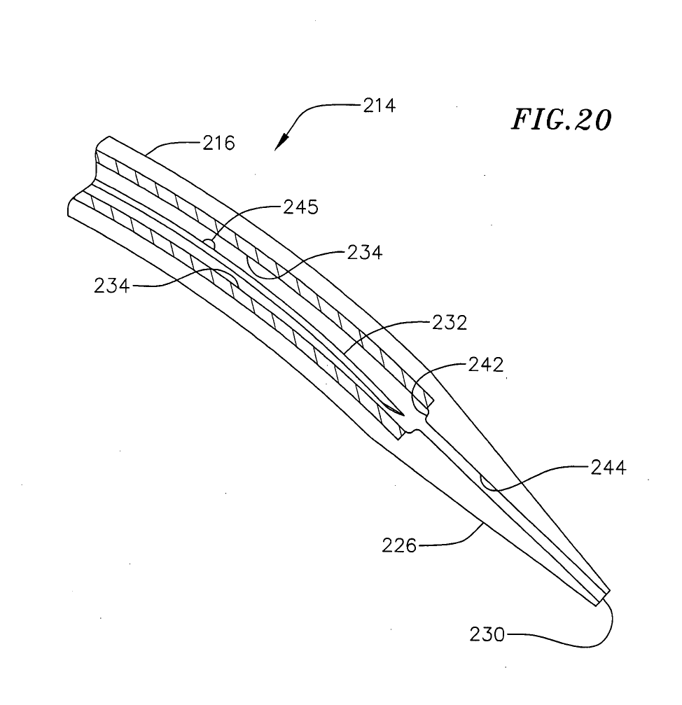

In accordance with another exemplary implementation of the present inventions, a needle may be used in combination with a shield or surface layer that is integral with one of the other components. Preferably, the shield or surface layer will be integral with an interior surface of a hollow element such as, for example, a dilator. As illustrated for example in FIGS. 18-20, a dilator 214 includes an elongate tube 216 with a proximal portion 218 having a female luer lock 220 and a distal portion 222. The distal portion 222 of the exemplary tube 216 has a region 224 with pre-formed curvature and a gradually converging surface 226 which terminates at the distal tip 228. The distal tip 228 defines an opening 230 (FIG. 20) through which a needle 232 may extend. The outer portion of the tube 216, which is preferably formed from conventional inert plastics, functions in a manner similar to conventional dilator tubes.

The exemplary dilator 214 illustrated in FIGS. 18-20 also includes an inner surface layer 234 formed integrally with, or secured on, an inside surface of the tube 216. The inner layer 234, which is located at least within the distal portion 222, forms a shroud that is positioned between the needle tip and the adjacent dilator tube surface. The inner surface layer 234 may extend over the same axial portion of the dilator that the needle tip will travel during normal procedures. For example, the longitudinal extent of the inner surface layer 234 may be at least the arc length of the curved area 224 and may extend somewhat beyond the longitudinal ends of the curved area where the tube 216 begins to straighten out. The inner layer 234, which can be formed as one or more longitudinal or circumferential segments or parts, may also extend the entire length of the tube 216.

A wide variety of shield or inner surface layer configurations may be employed. As illustrated in FIG. 19, the exemplary inner layer 234 includes an annular tube wall 236 for receiving and supporting the needle element 232. The tube wall 236 is supported within the dilator tube 216 by one or more (preferably four) support spokes or ribs 238 that extend outwardly from the tube wall outside surface 240 to the dilator tube inside surface 241. The annular tube wall 236 is also preferably coaxial with the dilator tube 216. Alternatively, the shield or inner surface layer may be formed from an appropriate coating, material deposit, co-extrusion or pultrusion, or other construction that is suitable for shielding the adjacent dilator surface from the needle tip. The shield or inner layer may also be a powder injection molded part or an insert molded part. With respect to materials, suitable shield and inner layer materials include metals (e.g. hypotubes), plastics, and fiber reinforced plastics such as carbon or glass reinforced plastics.

Referring to FIG. 20, the distal end of the exemplary inner surface layer 234 is substantially aligned with the proximal end of the converging surface 226. More specifically, the distal end of the inner surface layer 234 merges with, abuts or ends at a gradual shoulder 242 within the dilator tube 216 that converges inwardly toward a central axis of the dilator 214. The shoulder is gradual so that the needle slides easily along the exposed surface of the surface layer 234 and into a counterbore 244. The counter bore 244 extends from the shoulder 242 to the opening 230 and preferably has a substantially constant cross-sectional configuration from the shoulder to the opening. A needle element, such as the needle element 232, can extend into the counter bore and out through the opening 230 in order to, for example, cross the atrial septum. The needle preferably includes a raised surface area 245. The raised area 245 gives the widest point at the raised area 245 a dimension that is sufficiently greater than the inside diameter of the counterbore 244 to signal that the needle tip is advanced distally of the dilator the maximum desired amount, and even prevent further distal movement of the needle relative to the dilator.

The inner surface layer 234 is preferably no softer than the material from which the needle tip is formed, while the dilator tube 216 will preferably, but not necessarily, be formed from a plastic material that is softer and more flexible than either of the needle tip or the inner layer 234. The protection afforded by the inner layer 234 allows flexibility in selecting the configuration and material of the dilator tube 216. In the exemplary embodiment illustrated in FIGS. 18-20, the dilator tube 216 is about 0.020-0.030 inches thick, the annular tube wall 236 is about 0.020-0.030 inches thick, and the ribs 238 are about 0.040-0.060 inches thick. Where the inner surface layer 234 is a metal or other layer that rests directly against the inner surface of the dilator tube 216, the dilator tube wall may be thicker for structural support, and while the inner layer thickness may be about 0.001 inch to about 0.030 inch or more.

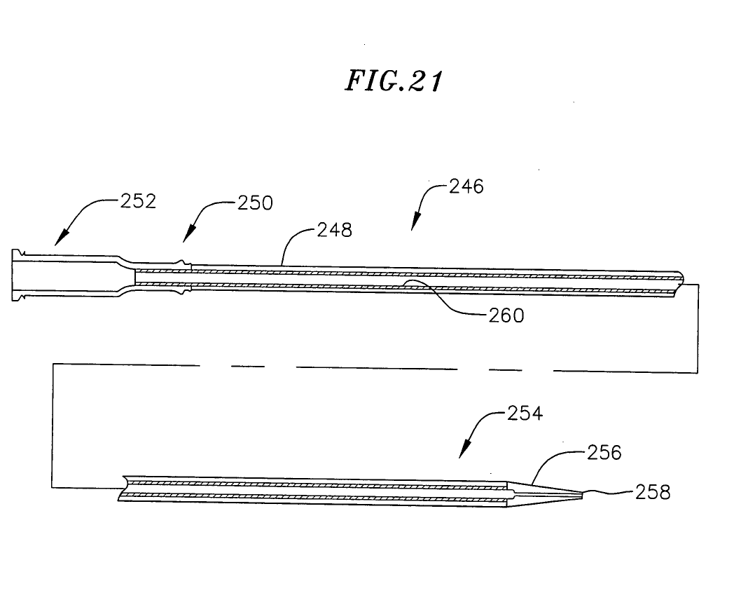

Turning to FIG. 21, an exemplary dilator 246 includes an elongate outer tube 248, which may have the same structure, function and configuration as the outer tube 216, with a proximal portion 250 that is secured to a female luer hub 252 and a distal portion 254. The distal portion 254 also has a converging surface 256 that terminates in a distal tip 258. Here, however, the exemplary dilator 246 does not have any pre-formed curvature. An inner surface layer shield 260 is included in at least a distal portion of the dilator 246 or may extend the entire length of the dilator. The inner layer 260 may have the same structures, function, configurations and can be formed from the same materials as the surface layer 234.

In use, the exemplary dilator 214 and needle element 232 illustrated in FIGS. 18-20 can be most easily used in conjunction with a guidewire system, while the dilator illustrated in FIG. 21 and a needle element can be most easily used in conjunction with a steerable catheter system. The dilator 214 and needle element 232 may be advanced with an introducer into the right atrium. The introducer and dilator may then be manipulated until the distal tip of the dilator is adjacent the desired location on the atrial septum. The needle element 232 may then be advanced across the atrial septum, followed by the dilator 214 and then the outer guide sheath of the introducer. The dilator 214 and needle element 232 may then be removed. The dilator 246 and a corresponding needle element can be advanced through a previously positioned introducer until the distal tip is adjacent to the atrial septum within the right atrium. After the position of the dilator 246 has been confirmed, the associated needle element may be advanced across the atrial septum, followed by the dilator and the outer guide sheath of the introducer. The dilator and needle combination can then be removed.

In another exemplary configuration of a dilator and needle combination shown in FIG. 22, which is generally represented by reference numeral 261, the dilator includes a distal portion 262 with a first bore 264, which terminates at a shoulder 266, and a counter bore 268 that defines an opening 270 in a rounded end surface 272. A needle segment 274 includes a preferably solid shaft 276 and a base 278. The base has an outside diameter that is sufficiently less than the inside diameter of the bore 264 to allow the needle to travel smoothly within the bore 264. A seal element 278a extends around the rim of the base 278 to provide a fluid seal between the base 278 and the bore 264. The shaft 276 extends into counter bore 268 and is sufficiently long to extend out the opening beyond the distal end of the dilator. The length of the shaft 276 is preferably such that the needle tip extends about 0.5-0.75 cm beyond the distal tip of the dilator, when the needle is fully advanced distally. The base 278 extends within the bore 264 and supports a spring 280 for biasing the shaft 276 proximally. The other end of the spring bears up against the shoulder 266.

The needle in the example shown in FIG. 22 can be advanced in a number of ways. In one example, the needle may be biased forward through a plunger-type action. A tube or other structure controlled proximally presses the needle distally through action at the proximal end of the dilator and tube or other structure. The spring 280 biases the needle proximally until the counter force from the tube is applied from the proximal end, pressing the needle distally against the bias of the spring. Similar needle motion can be achieved remotely using other pressure, for example fluid pressure. Fluid pressure can be applied to the proximal side of the base through the bore 264, against the bias of spring 280. Alternatively, the bore 264 can be configured to be closed proximally of the base 278, defining a chamber within which the needle can move, either with or without a counter-bias from a spring. In this configuration, the seal 278a is used to provide sufficient friction to be able to control needle movement. Needle movement occurs by increasing and decreasing the fluid pressure proximal of the base 278. When the pressure increases, the needle advances, and when the pressure is decreased, the needle retracts. Suitable conduits and surfaces can be incorporated into the dilator to develop the desired pressures to move the needle.

The puncture can also be made during the insertion and retraction process such that retention is not necessary.

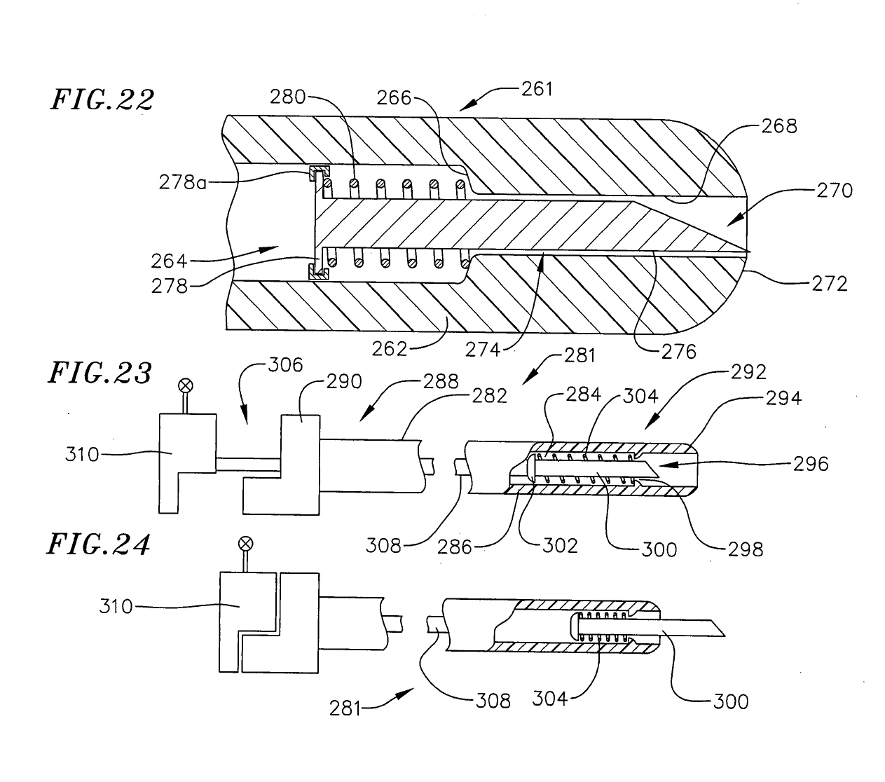

In another example of a dilator and needle combination, which is generally represented by reference numeral 281 in FIGS. 23 and 24, a dilator 282 includes an internal bore 284 within a dilator wall 286. The dilator proximal end portion 288 supports a luer hub 290 and the distal end portion 292 includes a converging surface 294 which defines an opening 296. A counter bore 298 is formed by a shoulder or ring on an inside surface of the dilator wall 286. The needle element, segment or tip 300 extends through the counter bore 298 and is sufficiently long to extend out of the dilator approximately 0.5-0.75 cm. The needle element 300 includes a support surface 302 for supporting a spring or other bias element 304 that pushes the needle tip proximally. A needle actuator 306, with a shaft or other actuating element 308 which is connected to a proximal handle 310, may be used to advance the needle element 300 distally through manipulation of the handle. When the handle 310 is advanced to the position shown in FIG. 24, the actuating element 308 pushes the needle element 300 against the spring 304 so that the needle element extends beyond the opening 296.

In the foregoing exemplary configurations of a needle and dilator, the needle may include shoulders or stop surfaces to indicate maximum preferred distal advance of the needle relative to the adjacent structures. At the proximal end of the several structures, one or more mechanisms may be used to releasably secure the needle to the outer sheath (or to the dilator, where no outer sheath is used). The mechanisms may be used to secure the needle in its distal-most position and/or secure the needle in its proximal-most position, as desired. Turning to FIG. 25, an engagement mechanism 312 is shown that holds the needle in its distal-most configuration relative to its outer sheath. The engagement mechanism 312 is shown in conjunction with a luer hub 314, similar to that described above with respect to FIG. 11, and a needle handle 316, also similar to that described above with respect to FIG. 11. The luer hub 314 supports an outer sheath similar to those described above. The hub 314 includes a surface defining a hole, recess or other engagement surface 318. The hole 318 receives bump, protrusion or other raised surface portion 320 on the needle to engage the bump and hold the needle relatively fixed with the outer sheath. The bump 320 is positioned near the end of a flexible and resilient tongue 322, which can be depressed to disengage the bump from the hole 318, so that the needle can be retracted easily.

Once the atrial septum has been punctured and the outer guide sheath advanced sufficiently into the left atrium, the dilator and needle combination can be withdrawn. A steerable catheter is then advanced along the outer guide sheath into the left atrium for the next phase of the procedure, which may include monitoring and sensing, diagnosis, or treatment, including tissue ablation, heart valve procedures and the like. Tissue ablation is described in U.S. Pat. No. 5,575,810, referenced above.

Having thus described several exemplary implementations of the invention, it will be apparent that various alterations and modifications can be made without departing from the inventions or the concepts discussed herein. Such operations and modifications, though not expressly described above, are nonetheless intended and implied to be within the spirit and scope of the inventions. Accordingly, the foregoing description is intended to be illustrative only.

1. An assembly, comprising:
a dilator including a hollow distal portion;
a sleeve movable within the hollow distal portion of the dilator and formed from a sleeve material having a first hardness; and
a needle movable within the sleeve and having a tip formed from a tip material having a second hardness no greater than the first hardness.

2. The assembly of claim 1 wherein the needle is a transseptal needle.

3. The assembly of claim 2 wherein the needle is formed from thin walled tubing.

4. The assembly of claim 1 wherein the distal portion of the dilator has an outer tapered surface.

5. The assembly of claim 1 wherein the distal portion of the dilator has an outer end surface that is rounded.

6. The assembly of claim 1 wherein the dilator is formed from plastic.

7. The assembly of claim 1 wherein at least some of the distal portion of the dilator is curved.

8. The assembly of claim 1 wherein the first hardness is greater than the second hardness.

9. The assembly of claim 1 wherein the sleeve material and the needle tip material are the same.

10. The assembly of claim 1 wherein the sleeve includes a distal portion with a converging surface.

11. The assembly of claim 1 wherein the sleeve includes a tapered distal portion.

12. The assembly of claim 1 wherein the sleeve includes a rounded distal portion.

13. The assembly of claim 1 wherein the sleeve comprises at least two portions.

14. The assembly of claim 13 wherein at least one of the at least two portions is a metal distal portion.

15. The assembly of claim 14 wherein the metal distal portion includes a converging surface.

16. The assembly of claim 14 wherein the metal distal portion extends around a portion of the needle.

17. The assembly of claim 14 wherein the metal distal portion of the sleeve includes a converging surface and the distal portion of the dilator has an internal surface that is complementary to the converging surface of the metal distal portion.

18. The assembly of claim 14 wherein the distal portion of the dilator has a curved portion and a portion of the metal distal portion of the sleeve is curved.

19. The assembly of claim 14 wherein at least one of the at least two portions is a plastic portion.

20. The assembly of claim 1 wherein the needle includes a proximal portion adjacent to the tip.

21. The assembly of claim 20 further comprising:
a bias element positioned and configured to bias the needle toward the distal portion of the sleeve.

22. The assembly of claim 21 wherein the bias element includes a spring.

23. The assembly of claim 21 further comprising:
a hold surface adapted to hold the needle in a first position.

24. The assembly of claim 1 wherein the sleeve and the needle include respective proximal portions each with at least one surface complementary to the surface on the other.

25. The assembly of claim 24 wherein complementary surfaces provide an interference fit.

26. The assembly of claim 24 wherein the complementary surfaces engage each other.

27. The assembly of claim 1 further comprising:
an introducer sheath configured to receive the dilator.

28. An assembly comprising:
a dilator including a distal portion with first and second segments, the first segment having a first hardness; and
a needle, movable within the dilator, including a tip having a second hardness no greater than the first hardness.

29. The assembly of claim 28 wherein the dilator is a transseptal dilator.

30. The assembly of claim 28 wherein the dilator defines a central axis and the first segment is closer to the central axis than the second segment.

31. The assembly of claim 30 wherein the first segment comprises a layer internal to the second segment.

32. The assembly of claim 30 wherein the first and second segments are concentric cylindrical segments.

33. The assembly of claim 28 wherein at least some of the distal portion of the dilator is curved.

34. The assembly of claim 33 wherein the curved portion of the dilator defines a proximal end and wherein a portion of the first segment is coextensive with proximal end of the curved portion of the dilator.

35. The assembly of claim 33 wherein the curved portion of the dilator and the first segment define respective proximal ends and the proximal end of the first segment is proximal of the proximal end of the curved portion of the dilator.

36. The assembly of claim 28 wherein the first and second segments extend longitudinally, the dilator defines a length, and the first segment extends substantially the length of the dilator.

37. The assembly of claim 28 wherein the first segment comprises a metal first segment.

38. The assembly of claim 28 wherein first segment comprises a plastic first segment.

39. The assembly of claim 28 wherein first hardness is harder than the second hardness.

40. The assembly of claim 28 wherein the first and second segments are concentric, the second segment is outside the first segment, and the second segment comprises a plastic second segment.

41. The assembly of claim 40 wherein the second segment is softer than the first segment.

42. The assembly of claim 28 wherein the dilator includes a distal portion having a converging surface.

43. The assembly of claim 42 wherein the needle includes a surface complementary to the converging surface of the dilator for contacting the convergence surface of the dilator.

44. The assembly of claim 42 wherein distal portion of the dilator includes first and second internal surfaces and the second internal surface is closer to the needle than the first internal surface.

45. The assembly of claim 28 wherein the needle is formed from hypotube.

46. The assembly of claim 28 further comprising:
an introducer sheath configured to receive the dilator.

47. An assembly, comprising:
a dilator tube having a proximal portion and a distal portion, the distal portion including a converging surface and a distal end opening;
a transport tube having a proximal portion, a distal portion and a distal end, the transport distal end defining a transport distal end opening, the transport tube being located within the dilator tube and configured such that the distal portion of the transport tube may be positioned adjacent to the distal portion of the dilator tube; and
a needle within the transport tube and movable relative to the transport tube.

48. The assembly of claim 47 wherein the dilator tube defines a first transverse cross-sectional area proximal to the distal end opening of the dilator tube and the distal end opening of the dilator tube defines a second cross-sectional area less than the first cross-sectional area.

49. The assembly of claim 48 wherein the distal portion of the transport tube defines an outer transverse cross-sectional area greater than the second cross-sectional area.

50. The assembly of claim 49 wherein the transport tube has a bevel on an outer surface and the dilator tube has an inner surface with a shoulder complementary to the bevel.

51. The assembly of claim 50 wherein the distal end of the transport tube does not extend outside the dilator tube when the bevel abuts the shoulder.

52. The assembly of claim 50 wherein the distal end of the transport tube extends outside the dilator tube when the bevel abuts the shoulder.

53. The assembly of claim 47 wherein the transport tube includes an outer wall with straight sides.

54. The assembly of claim 53 wherein the transport tube includes an inner wall that converges to a needle opening that extends around the needle.

55. The assembly of claim 54 wherein the needle comprises a hypotube.

56. The assembly of claim 54 wherein the transport tube and dilator tube are respectively configured such that the distal end of the transport tube is prevented from reaching the distal end opening of the dilator tube.

57. The assembly of claim 54 wherein the distal end opening of the transport tube is smaller than the distal end opening of the dilator.

58. The assembly of claim 47 wherein the needle and transport tube are formed from respective hypotubes.

59. The assembly of claim 58 wherein the transport tube is approximately the same length as the dilator tube.

60. The assembly of claim 58 wherein the transport tube extends past the distal end of the dilator.

61. A method of assembling a needle/dilator combination, the method comprising the steps of:
positioning a dilator, having a proximal portion, a distal portion and a distal opening, such that the proximal portion is accessible;
introducing a transport tube formed from a first material into the proximal portion of the dilator;
passing a needle formed from a second material no harder than the first material into the transport tube; and
advancing the needle within the transport tube until the needle extends beyond the dilator distal opening.

62. The method of claim 61 wherein the step of introducing a transport tube into the dilator occurs after the step of passing a needle into the transport tube.

63. The method of claim 62 wherein the step of advancing the needle occurs after the step of passing the needle into the transport tube.

64. The method of claim 61 further comprising the step of:
advancing the transport tube within the dilator until a distal portion of the transport tube is adjacent to the distal portion of the dilator.

65. The method of claim 64 wherein the step of advancing the needle occurs after the step of advancing the transport tube within the dilator.

66. The method of claim 61 further comprising the step of:
introducing the dilator into a sheath.

67. The method of claim 61 further comprising the step of:
passing the transport tube over a guidewire.

68. An assembly, comprising:
a dilator defining a distal portion;
a needle movable within the dilator and having a needle tip formed from a material having a first material configuration; and
a surface layer between the dilator distal portion and the needle, the surface layer having a second material configuration different than the first material configuration.

69. The assembly of claim 68 wherein the first material configuration is a first hardness and wherein the second material configuration is a second hardness greater than the first hardness.

70. The assembly of claim 68 wherein the first material configuration is a chemical composition and the second material configuration is a chemical composition different than the chemical composition of the first material configuration.

71. The assembly of claim 68 wherein the surface layer is integral with the dilator distal portion.

72. The assembly of claim 68 wherein the surface layer is movable relative to the dilator and to the needle.

73. The assembly of claim 72 wherein the surface layer includes a tubular member.

74. The assembly of claim 72 wherein the dilator comprises a plastic dilator, the surface layer is formed from a hypotube, and the needle is formed from a hypotube.
